(19) United States
(12) Patent Application Publication
(10) Pub. No.: US 2025/0281279 A1
(43) Pub. Date: Sep. 11, 2025

(54) ARTIFICIAL EPIGLOTTIS, AND SWALLOWING AND BREATHING SWITCHING DEVICE

(71) Applicant: Murata Manufacturing Co., Ltd., Nagaokakyo-shi (JP)

(72) Inventor: Michiyo YAMAMOTO, Nagaokakyo-shi (JP)

(21) Appl. No.: 19/216,950

(22) Filed: May 23, 2025

Related U.S. Application Data
(63) Continuation of application No. PCT/JP2023/042888, filed on Nov. 30, 2023.

(30) Foreign Application Priority Data
Dec. 1, 2022 (JP) 2022-192620

(51) Int. Cl. A61F 2/20; A61F 2/48

(52) U.S. Cl. CPC A61F 2/203; A61F 2/482; A61F 2250/0002

(57) ABSTRACT

An artificial epiglottis is provided that includes a membrane-like piezoelectric element that bends depending on a voltage; and a first driving electrode that applies the voltage to the piezoelectric element. The piezoelectric element is structurally configured to be disposed on a larynx. Based on the applied voltage, the piezoelectric element bends to either a first shape in which an entrance of a trachea is blocked or to a second shape in which the entrance of the trachea is opened.

### CROSS REFERENCE TO RELATED APPLICATIONS

This application is a continuation of International Application No. PCT/JP2023/042888, filed Nov. 30, 2023, which claims priority to Japanese Patent Application No. 2022-192620, filed Dec. 1, 2022, the contents of each of which are hereby incorporated by reference in their entirety.

### TECHNICAL FIELD

The present disclosure relates to an artificial epiglottis and an artificial technique of switching between swallowing and breathing using the artificial epiglottis.

### BACKGROUND

The number of patients with swallowing dysfunction is increasing due to the super-aging society. Swallowing dysfunction may cause aspiration. Aspiration is a phenomenon in which something that should enter the esophagus from the mouth enters the respiratory tract and has various adverse effects on the human body.

Japanese Unexamined Patent Application Publication No. 2020-54528 describes a swallowing movement assisting orthosis. The orthosis described therein assists the thyroid cartilage to be mechanically pushed upward from a front-lower position in the laryngeal elevation movement that occurs during the swallowing movement pharyngeal stage.

Moreover, Japanese Unexamined Patent Application Publication No. 11-500339 describes a method for treating dysphagia by electrical stimulation. The method described therein stimulates muscles by electrical stimulation to assist swallowing.

However, the conventional techniques described above provide swallowing assistance and training. Therefore, it is difficult to sufficiently suppress aspiration with conventional techniques described therein.

### SUMMARY OF THE INVENTION

In view of the foregoing, it is an object of the present disclosure to provide a device that is configured to suppress aspiration more reliably.

In particular, an artificial epiglottis according to an exemplary embodiment is provided that includes a membrane-like piezoelectric element configured to bend depending on a voltage; and a driving electrode configured to apply the voltage to the piezoelectric element. The piezoelectric element is structurally configured to be disposed on a larynx. The piezoelectric element is, based on the voltage applied by the driving electrode, configured to become a first shape in which an entrance of a trachea is blocked and or a second shape in which the entrance of the trachea is opened.

With such a configuration, the artificial epiglottis is provided that is configured to switch between a state in which the entrance of the trachea is blocked and a state in which the entrance of the trachea is open by using the bending of the piezoelectric element. Since the artificial epiglottis is formed by the piezoelectric element, switching speed is fast, switching accuracy is high, and posture holding ability of each state is high. Therefore, swallowing and breathing can be switched more reliably.

According to the exemplary aspects of the present disclosure, aspiration is suppressed more reliably by the artificial epiglottis disclosed described herein.

### BRIEF DESCRIPTION OF DRAWINGS

FIG. 1 is an external perspective view showing an example of a configuration of a swallowing and breathing switching device according to a first exemplary embodiment.

FIGS. 2(A) and 2(B) are views showing a configuration of the swallowing and breathing switching device according to the first exemplary embodiment.

FIG. 3(A) is a schematic view showing a breathing state in which an artificial epiglottis and the swallowing and breathing switching device according to the first exemplary embodiment are worn, and FIG. 3(B) is a schematic view showing a swallowing state in which the artificial epiglottis and the swallowing and breathing switching device according to the first exemplary embodiment are worn.

FIGS. 4(A) and 4(B) are views showing a configuration of a swallowing and breathing switching device according to a second exemplary embodiment.

FIG. 5 is a view showing a configuration of a swallowing and breathing switching device according to a third exemplary embodiment.

FIG. 6 is a view showing a configuration of a swallowing and breathing switching device according to a fourth exemplary embodiment.

FIG. 7 is a view showing a configuration of a swallowing and breathing switching device according to a fifth exemplary embodiment.

FIG. 8 is a view showing a configuration of a swallowing and breathing switching device according to a sixth exemplary embodiment.

FIG. 9 is a view showing a configuration of a swallowing and breathing switching device according to a seventh exemplary embodiment.

FIG. 10 is a view showing a configuration of a swallowing and breathing switching device according to an eighth exemplary embodiment.

FIG. 11 is a view showing a configuration of a swallowing and breathing switching device according to a ninth exemplary embodiment.

FIG. 12(A), FIG. 12(B), and FIG. 12(C) are perspective views each showing a configuration example of an artificial epiglottis according to a tenth exemplary embodiment.

FIG. 13 is a schematic view showing a curved state of the artificial epiglottis shown in FIG. 12(A).

FIG. 14 is a view showing a configuration of a swallowing and breathing switching device according to an eleventh exemplary embodiment.

### DETAILED DESCRIPTION OF EMBODIMENTS

### First Exemplary Embodiment

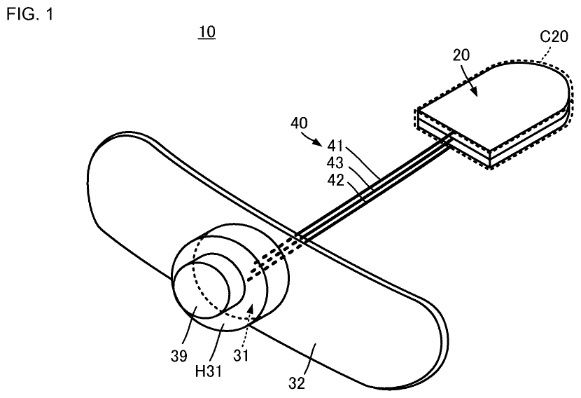

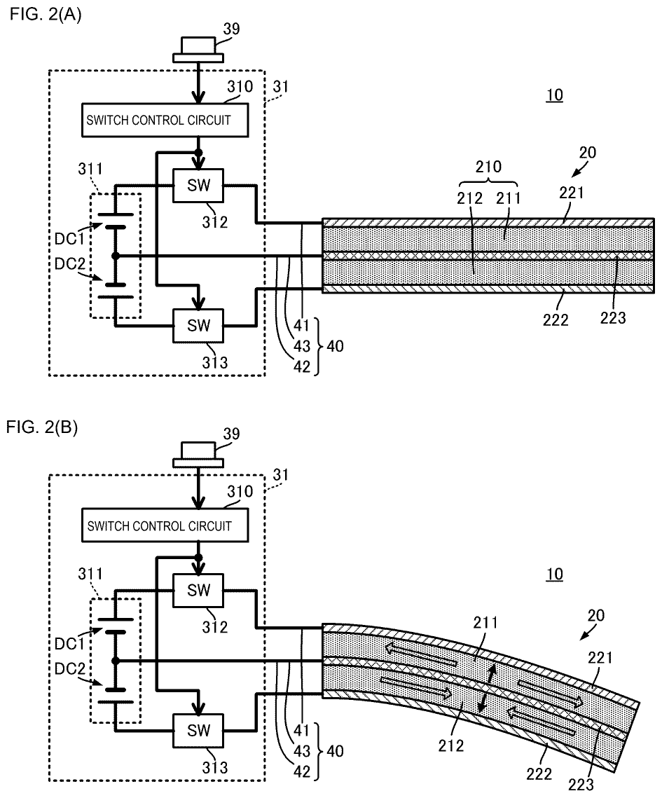

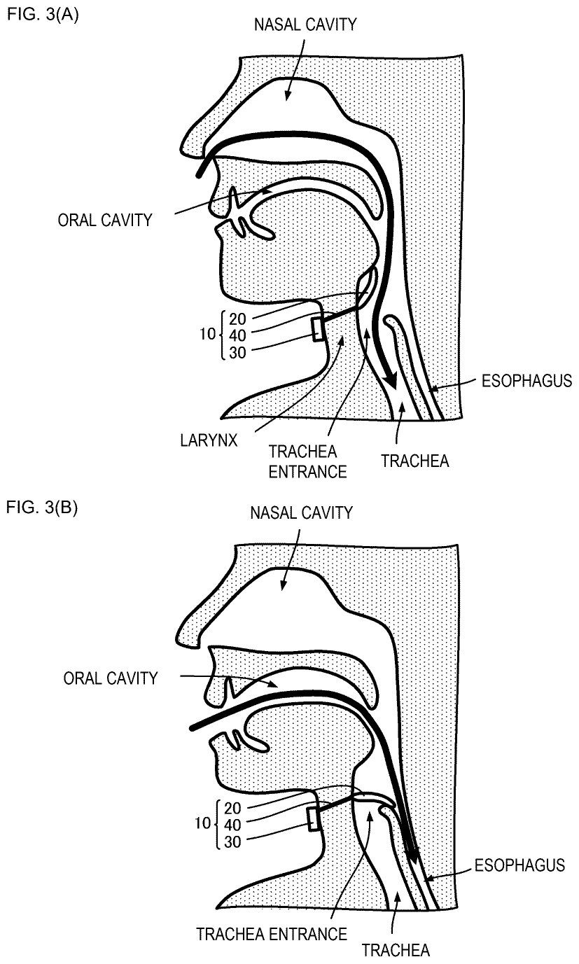

An artificial epiglottis and a swallowing and breathing switching device according to a first exemplary embodiment will be described with reference to the drawings. FIG. 1 is an external perspective view of the swallowing and breathing switching device according to the first exemplary embodiment. FIGS. 2(A) and 2(B) are views showing a configuration of the swallowing and breathing switching device according to the first exemplary embodiment, wherein FIG. 2(A) shows a first shape of the artificial epiglottis and FIG. 2(B) shows a second shape of the artificial epiglottis. FIG. 3(A) is a schematic view showing a breathing state in which the artificial epiglottis and the swallowing and breathing switching device according to the first exemplary embodiment are worn, and FIG. 3(B) is a schematic view showing a swallowing state in which the artificial epiglottis and the swallowing and breathing switching device according to the first exemplary embodiment are worn.

### Physical Configuration of Swallowing and Breathing Switching Device 10

As shown in FIGS. 1, 2(A) and 2(B), a swallowing and breathing switching device 10 includes an artificial epiglottis 20, a drive signal generating unit 31, an orthosis 32, a physical switch 39, and a cable 40.

The drive signal generating unit 31 is configured by using at least one of an electric circuit element and an electronic circuit element. The drive signal generating unit 31 is incorporated in a predetermined housing H31.

The physical switch 39 is provided in the housing H31. The physical switch 39 is electrically connected to the drive signal generating unit 31.

The orthosis 32 has a belt-like shape. The housing H31 is fixed to the front side of the orthosis 32.

The cable 40 has a first cable 41, a second cable 42, and a third cable 43. The first cable 41, the second cable 42, and the third cable 43 are each a conductive wire whose peripheral surface is covered with an insulating material, for example. The insulating material of the first cable 41, the second cable 42, and the third cable 43 may be a biocompatible material in an exemplary aspect.

As shown, one end of the cable 40 (the first cable 41, the second cable 42, and the third cable 43) is connected to the drive signal generating unit 31. The cable 40 (the first cable 41, the second cable 42, and the third cable 43) is disposed so as to extend from the back surface of the orthosis 32. The other end of the cable 40 (the first cable 41, the second cable 42, and the third cable 43) is connected to the artificial epiglottis 20.

The artificial epiglottis 20 includes a piezoelectric element 210, a first driving electrode 221, a second driving electrode 222, and a third driving electrode 223. The piezoelectric element 210 includes a first piezoelectric element 211 and a second piezoelectric element 212.

The first piezoelectric element 211 and the second piezoelectric element 212 have a flat membrane-like shape. The main material of the first piezoelectric element 211 and the second piezoelectric element 212 is, for example, polylactic acid. The first piezoelectric element 211 and the second piezoelectric element 212 are configured to extend or contract in a direction parallel to the flat membrane surfaces thereof depending on the voltage applied thereto.

In the exemplary aspect, the first piezoelectric element 211 and the second piezoelectric element 212 are laminated together so that the flat membrane surfaces thereof are parallel to each other.

The first driving electrode 221 is disposed on the flat membrane surface of the first piezoelectric element 211 on a side opposite to the surface facing the second piezoelectric element 212. The second driving electrode 222 is disposed on the flat membrane surface of the second piezoelectric element 212 on a side opposite to the surface facing the first piezoelectric element 211. The third driving electrode 223 is disposed between the first piezoelectric element 211 and the second piezoelectric element 212. Thus, the first piezoelectric element 211 is sandwiched between the first driving electrode 221 and the third driving electrode 223. The second piezoelectric element 212 is sandwiched between the second driving electrode 222 and the third driving electrode 223.

In the artificial epiglottis 20 having this configuration according to the exemplary aspect, the first piezoelectric element 211 does not expand or contract when the third driving electrode 223 and the first driving electrode 221 have the same potential. Similarly, the second piezoelectric element 212 does not expand or contract when the third driving electrode 223 and the second driving electrode 222 have the same potential. Therefore, the artificial epiglottis 20 is not bent (e.g., curved) as shown in FIG. 2(A). This state is the first shape of the artificial epiglottis 20.

On the other hand, when a voltage is applied to the first piezoelectric element 211 so that the first driving electrode 221 has a positive potential with respect to the third driving electrode 223, the first piezoelectric element 211 will extend in a direction parallel to the flat membrane surface. When a voltage is applied to the second piezoelectric element 212 so that the first driving electrode 221 has a positive potential with respect to the third driving electrode 223, the second piezoelectric element 212 will contract in a direction parallel to the flat membrane surface.

In the exemplary aspect, the first piezoelectric element 211 and the second piezoelectric element 212 are laminated together in a direction orthogonal to the flat membrane surface. As described above, when the first piezoelectric element 211 extends and the second piezoelectric element 212 contracts, the artificial epiglottis 20 composed of a laminated structure of the first piezoelectric element 211 and the second piezoelectric element 212 bends (e.g., curves) so as to bend toward the second piezoelectric element 212 as shown in FIG. 2(B). This state is the second shape of the artificial epiglottis 20. At this time, the extension due to the voltage applied to the first piezoelectric element 211 and the contraction due to the voltage applied to the second piezoelectric element 212 are synergized, so that the artificial epiglottis 20 bends larger than a case in which only the first piezoelectric element 211 or only the second piezoelectric element 212 is provided.

According to an exemplary aspect, the first

piezoelectric element 211, the second piezoelectric element 212, the first driving electrode 221, the second driving electrode 222, and the third driving electrode 223 forming the artificial epiglottis 20 are preferably made of a biocompatible material. However, as shown in FIG. 1, the artificial epiglottis 20 may include a cover C20. In this regard, the cover C20 is flexible enough not to inhibit the bending of the artificial epiglottis 20. When the cover C20 is provided, the first piezoelectric element 211, the second piezoelectric element 212, the first driving electrode 221, the second driving electrode 222, and the third driving electrode 223 do not have to be made of a biocompatible material as long as the cover C20 is made of a biocompatible material.

### Electrical Configuration of swallowing and Breathing Switching Device 10

The drive signal generating unit 31 of the swallowing and breathing switching device 10 includes a power supply circuit 311, an electric switch 312, an electric switch 313, and a switch control circuit 310.

The power supply circuit 311 includes a direct-current power supply DC1 and a direct-current power supply DC2. The direct-current power supply DC1 and the direct-current power supply DC2 are, for example, primary batteries or secondary batteries.

The negative electrode of the direct-current power supply DC1 and the negative electrode of the direct-current power supply DC2 are connected to each other. The node of the negative electrode of the direct-current power supply DC1 and the negative electrode of the direct-current power supply DC2 are connected to the third driving electrode 223 of the artificial epiglottis 20 through the third cable 43.

As shown, the positive electrode of the direct-current power supply DC1 is connected to the electric switch 312. The electric switch 312 is connected to the first driving electrode 221 of the artificial epiglottis 20 through the first cable 41.

The positive electrode of the direct-current power supply DC2 is connected to the electric switch 313. The electric switch 313 is connected to the second driving electrode 222 of the artificial epiglottis 20 through the second cable 42.

The switch control circuit 310 is connected to the physical switch 39 and connected to the electric switch 312 and the electric switch 313.

The switch control circuit 310 generates a switch control signal for the electric switch 312 and the electric switch 313 according to the operating state of the physical switch 39. The electric switch 312 and the electric switch 313 are configured to switch between open and short-circuit according to the switch control signal.

For example, in a state in which the physical switch 39 is not operated, the switch control circuit 310 does not output the switch control signal to the electric switch 312 and the electric switch 313. In such a case, the electric switch 312 and the electric switch 313 are in an open state. Therefore, no direct-current voltage for driving is applied to the artificial epiglottis 20. As a result, the artificial epiglottis 20 becomes the first shape shown in FIG. 2(A).

On the other hand, in a state in which the physical switch 39 is operated, the switch control circuit 310 is configured to generate a switch control signal for switching between a Hi state and a Low state at a predetermined period, and to output the generated switch control signal to the electric switch 312 and the electric switch 313.

The electric switch 312 and the electric switch 313 are in a short-circuit state when the switch control signal is in the Hi state and are in an open state when the switch control signal is in the Low state.

When the electric switch 312 and the electric switch 313 are in the open state, no direct-current voltage for driving is applied to the artificial epiglottis 20 (an example of a first state of the drive signal). Therefore, the artificial epiglottis 20 becomes the first shape shown in FIG. 2(A).

When the electric switch 312 and the electric switch 313 are in the short-circuit state, a direct-current voltage for driving is applied to the artificial epiglottis 20 (an example of a second state of the drive signal). Therefore, the artificial epiglottis 20 becomes the second shape shown in FIG. 2(B).

Thus, the swallowing and breathing switching device 10 can be configured to switch between the first shape in which the artificial epiglottis 20 is not bent and the second shape in which the artificial epiglottis 20 is bent.

### Wearing on Human Body and Practical Wearing Condition

As shown in FIGS. 3(A) and 3(B), the artificial epiglottis 20 is disposed on the larynx of a body. More specifically, one end of the artificial epiglottis 20 in the direction in which the first piezoelectric element 211 and the second piezoelectric element 212 expand and contract is fixed to the larynx. The other end of the artificial epiglottis 20 in the direction in which the first piezoelectric element 211 and the second piezoelectric element 212 expand and contract is a free end not fixed to the larynx.

In a state in which no voltage is applied to the artificial epiglottis 20, the artificial epiglottis 20 is disposed so as not to block the entrance of the trachea, as shown in FIG. 3(A). Further, in a state in which a voltage is applied to the artificial epiglottis 20, the artificial epiglottis 20 is disposed so as to block the entrance of the trachea, as shown in FIG. 3(B).

The housing H31, in which the drive signal generating unit 31 is incorporated, is worn on the front neck of the body by the orthosis 32. The cable 40 (the first cable 41, the second cable 42, and the third cable 43) is disposed through the larynx, and connects the drive signal generating unit 31 and the artificial epiglottis 20.

In such a state, when the wearer presses the physical switch 39, the switch control circuit 310 is driven. The switch control circuit 310 is configured to switch between opening and short-circuiting of the electric switch 312 and the electric switch 313.

More specifically, when the drive signal generating unit 31 (the switch control circuit 310) opens the electric switch 312 and the electric switch 313, the artificial epiglottis 20 is configured to become (e.g., change or bend) the first shape, so that the entrance of the trachea is not blocked, as shown in FIG. 3(A). Therefore, air from the nasal cavity enters the trachea, so that breathing is performed without any problem.

On the other hand, when the drive signal generating unit 31 (the switch control circuit 310) short-circuits the electric switch 312 and the electric switch 313, the artificial epiglottis 20 is configured to become (e.g., change or bend) the second shape, so that the entrance of the trachea is blocked by the artificial epiglottis 20, as shown in FIG. 3(B). Therefore, food (drink) from the oral cavity enters the esophagus, so that swallowing is performed without any problem.

The above configuration shows an aspect of switching between the opening and short-circuiting of the electric switch 312 and the electric switch 313, in other words, between the first shape and the second shape of the artificial epiglottis 20, at a predetermined period. However, the swallowing and breathing switching device 10 may selectively open and short-circuit the electric switch 312 and the electric switch 313. Specifically, the swallowing and breathing switching device 10 can be configured to perform control to short-circuit the electric switch 312 and the electric switch 313 only when swallowing is performed. The swallowing and breathing switching device 10 can be configured to perform control to open the electric switch 312 and the electric switch 313 when swallowing is not performed (i.e., when breathing).

As described above, by providing the artificial epiglottis 20 and the swallowing and breathing switching device 10, aspiration is suppressed more reliably.

Since aspiration is suppressed more reliably, aspiration pneumonia is also suppressed. Further, since aspiration is suppressed more reliably, food can be taken from the mouth without the need for a gastrostomy or enterostomy. Further, since aspiration can be suppressed more reliably, a tracheostomy is not required. Further, since aspiration is suppressed more reliably, food restrictions such as having to make food thick can be eliminated.

By providing the artificial epiglottis 20 and the swallowing and breathing switching device 10, tracheal cannula-related problems (such as pain during regular replacement and choking due to blockage in the tracheal cannula) can be prevented. Further, by providing the artificial epiglottis 20 and the swallowing and breathing switching device 10, rehabilitation to train the periphery of the larynx is not required.

Further, in the swallowing and breathing switching device 10, the first piezoelectric element 211 and the second piezoelectric element 212 are used in the artificial epiglottis 20. With such a configuration, the swallowing and breathing switching device 10 can be configured to switch (e.g., adapted or bent) between the first shape and the second shape of the artificial epiglottis 20 at high speed. In addition, the artificial epiglottis 20 can stably hold the first shape and the second shape. In addition, the artificial epiglottis 20 can be realized with low power consumption. In addition, electromagnetic noise generated by the artificial epiglottis 20 is suppressed. In addition, the artificial epiglottis 20 can be formed in a small size and thin shape so as to be easily disposed at a desired position of the larynx. In addition, the artificial epiglottis 20 can be realized with a simple configuration.

As shown in FIG. 1(A), for example, the artificial epiglottis 20 has two piezoelectric elements (the first piezoelectric element 211 and the second piezoelectric element 212) laminated together. Further, the first piezoelectric element 211 and the second piezoelectric element 212 have opposite expansion and contraction directions (e.g., displacement directions) when a voltage is applied. Therefore, the bending effect caused by the first piezoelectric element 211 and the bending effect caused by the second piezoelectric element 212 are synergized. Thus, the artificial epiglottis 20 can realize a larger bending amount. As a result, the artificial epiglottis 20 can increase the difference between the first shape in which the entrance of the trachea is not blocked and the second shape in which the entrance of the trachea is blocked, so that aspiration is suppressed more reliably.

In the present embodiment, an aspect has been described in which the piezoelectric element 210 uses a multilayer body of the first piezoelectric element 211 and the second piezoelectric element 212 (e.g., a bimorph drive). However, the piezoelectric element 210 may alternatively be provided with at least one of the first piezoelectric element 211 and the second piezoelectric element 212 (e.g., an unimorph drive).

### Second Exemplary Embodiment

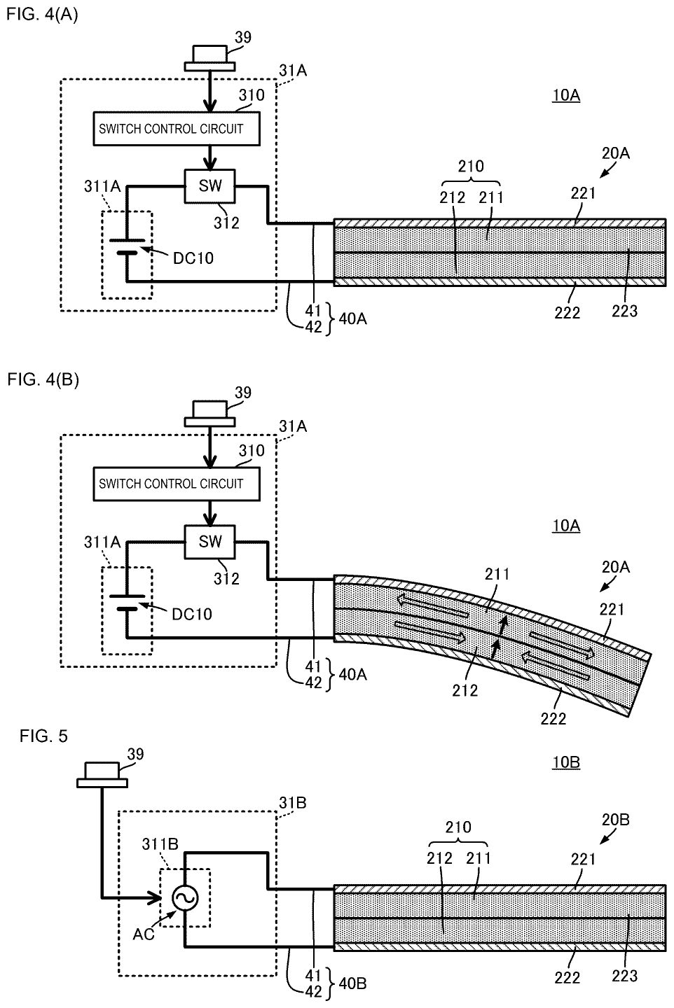

An artificial epiglottis and a swallowing and breathing switching device according to a second exemplary embodiment will be described with reference to the drawings. FIGS. 4(A) and 4(B) are views showing a configuration of a swallowing and breathing switching device according to a second exemplary embodiment, wherein FIG. 4(A) shows a first shape of the artificial epiglottis and FIG. 4(B) shows a second shape of the artificial epiglottis.

As shown in FIGS. 4(A) and 4(B), a swallowing and breathing switching device 10A according to the second embodiment is different from the swallowing and breathing switching device 10 according to the first embodiment in that the swallowing and breathing switching device 10A includes an artificial epiglottis 20A and a drive signal generating unit 31A. The other configurations of the swallowing and breathing switching device 10A are the same as those of the swallowing and breathing switching device 10, and descriptions of the same parts will be omitted.

The swallowing and breathing switching device 10A includes the artificial epiglottis 20A, the drive signal generating unit 31A, and a cable 40A. The cable 40A includes a first cable 41 and a second cable 42.

The artificial epiglottis 20A includes a first piezoelectric element 211, a second piezoelectric element 212, a first driving electrode 221, and a second driving electrode 222. The first piezoelectric element 211 and the second piezoelectric element 212 are laminated in contact with each other. The first driving electrode 221 is disposed on a surface of the first piezoelectric element 211 on a side opposite to the contact surface of the second piezoelectric element 212. The second driving electrode 222 is disposed on a surface of the second piezoelectric element 212 on a side opposite to the contact surface of the first piezoelectric element 211.

When a voltage is applied to the first piezoelectric element 211 so that the first driving electrode 221 has a positive potential with respect to the second driving electrode 222, the first piezoelectric element 211 will extend in a direction parallel to the flat membrane surface. When a voltage is applied to the second piezoelectric element 212 so that so that the first driving electrode 221 has a positive potential with respect to the second driving electrode 222, the second piezoelectric element 212 will contract in a direction parallel to the flat membrane surface.

The drive signal generating unit 31A includes a power supply circuit 311A, an electric switch 312, and a switch control circuit 310. The power supply circuit 311A includes a direct-current power supply DC10. The positive electrode of the direct-current power supply DC10 is connected to the first driving electrode 221 through the electric switch 312 and the first cable 41. The negative electrode of the direct-current power supply DC10 is connected to the second driving electrode 222 through the second cable 42.

With such a configuration, when the electric switch 312 is short-circuited, a voltage from the direct-current power supply DC10 is applied to the artificial epiglottis 20A (an example of a first state of the drive signal). When the electric switch 312 is opened, no voltage is applied to the artificial epiglottis 20A (an example of a second state of the drive signal).

With such a configuration, the swallowing and breathing switching device 10A can be configured to switch between a state in which the entrance of the trachea is not blocked and a state in which the entrance of the trachea is blocked, in the same manner as the swallowing and breathing switching device 10, so that aspiration is suppressed more reliably.

### Third Exemplary Embodiment

An artificial epiglottis and a swallowing and breathing switching device according to the third exemplary embodiment will be described with reference to the drawings. FIG. 5 is a view showing a configuration of the swallowing and breathing switching device according to the third exemplary embodiment.

As shown in FIG. 5, a swallowing and breathing switching device 10B according to the third embodiment is different from the swallowing and breathing switching device 10 according to the first embodiment in that the swallowing and breathing switching device 10B includes an artificial epiglottis 20B, a drive signal generating unit 31B, and a cable 40B. The artificial epiglottis 20B is the same as the artificial epiglottis 20B according to the second embodiment, and the cable 40B is the same as the cable 40A according to the second embodiment.

The drive signal generating unit 31B includes a power supply circuit 311B. The power supply circuit 311B includes an alternating-current power supply AC. One output terminal of the alternating-current power supply AC is connected to the first driving electrode 221 through the first cable 41. The other output terminal of the alternating-current power supply AC is connected to the second driving electrode 222 through the second cable 42. The physical switch 39 is connected to the power supply circuit 311B.

The power supply circuit 311B receives an operation signal from the physical switch 39 and drives the alternating-current power supply AC. The alternating-current power supply AC applies an alternating-current voltage to the artificial epiglottis 20B. The alternating-current voltage is a voltage that alternates between a Hi state (an example of a first state of the drive signal) and a Low state (an example of a second state of the drive signal).

For example, the artificial epiglottis 20B becomes a first shape in which it does not bend when the alternating-current voltage is in the Low state and becomes a second shape in which it bends when the alternating-current voltage is in the Hi state.

For example, the period of the alternating-current

voltage corresponds to the period of salivation during sleep, the period of breathing and/or the like.

With such a configuration, the swallowing and breathing switching device 10B can be configured to switch between a state in which the entrance of the trachea is not blocked and a state in which the entrance of the trachea is blocked, in the same manner as the swallowing and breathing switching device 10, so that aspiration is suppressed more reliably.

### Fourth Exemplary Embodiment

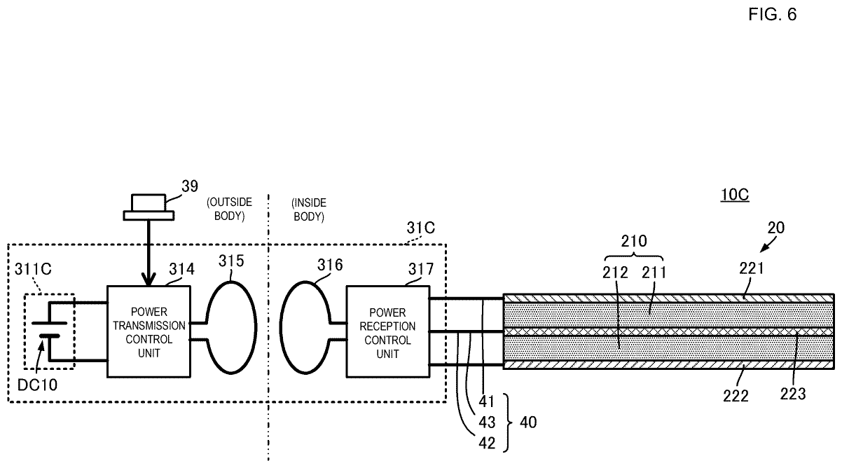

An artificial epiglottis and a swallowing and breathing switching device according to a fourth exemplary embodiment will be described with reference to FIG. 6. FIG. 6 is a view showing a configuration of the swallowing and breathing switching device according to the fourth exemplary embodiment.

As shown in FIG. 6, a swallowing and breathing switching device 10C according to the fourth embodiment is different from the swallowing and breathing switching device 10 according to the first embodiment in that the swallowing and breathing switching device 10C includes a drive signal generating unit 31C. The other configurations of the swallowing and breathing switching device 10C are the same as those of the swallowing and breathing switching device 10, and descriptions of the same parts will be omitted.

The swallowing and breathing switching device 10C includes the drive signal generating unit 31C. The drive signal generating unit 31C includes a power supply circuit 311C, a power transmission control unit 314, a power transmission coil 315, a power reception coil 316, and a power reception control unit 317. The power supply circuit 311C, the power transmission control unit 314, and the power transmission coil 315 are disposed outside the body. The power reception coil 316 and the power reception control unit 317 are disposed inside the body.

The power supply circuit 311C includes a direct-current power supply DC10. The direct-current power supply DC10 is connected to the power transmission control unit 314. The power transmission control unit 314 is connected to the power transmission coil 315.

The power reception coil 316 is connected to the power reception control unit 317. The power reception control unit 317 is connected to the artificial epiglottis 20 through a cable 40 (a first cable 41, a second cable 42, and a third cable 43).

The power transmission control unit 314 receives an operation signal from the physical switch 39, converts a direct-current voltage from the direct-current power supply DC10, and supplies an alternating-current power transmission current to the power transmission coil 315. The power transmission coil 315 is configured to excite an alternating magnetic field by the alternating-current power transmission current.

The power reception coil 316 is coupled to the alternating magnetic field to generate an alternating-current power reception current. The power reception control unit 317 rectifies the power reception current, generates a direct-current voltage, and supplies the direct-current voltage to the artificial epiglottis 20.

As described above, the drive signal generating unit 31C employs a wireless power supply system.

With such a configuration, the swallowing and breathing switching device 10C can be configured to switch between a state in which the entrance of the trachea is not blocked and a state in which the entrance of the trachea is blocked, in the same manner as the swallowing and breathing switching device 10, so that aspiration is suppressed more reliably.

Note that the power reception control unit 317 may include a storage battery. By charging the storage battery, the swallowing and breathing switching device 10C can supply a voltage to the artificial epiglottis 20 even in a state in which no unit is provided outside the body in the drive signal generating unit 31C.

### Fifth Exemplary Embodiment

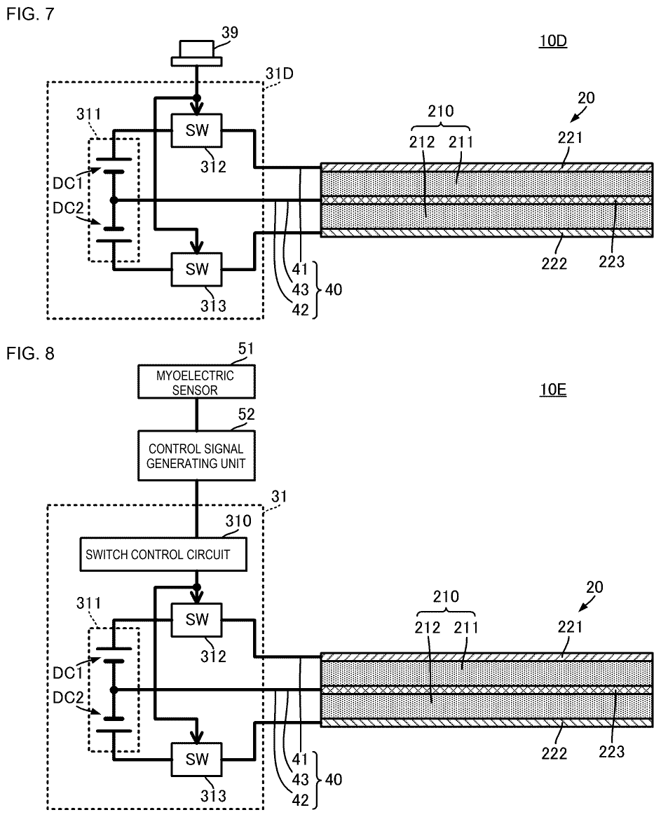

An artificial epiglottis and a swallowing and breathing switching device according to a fifth exemplary embodiment will be described with reference to the drawings. FIG. 7 is a view showing a configuration of the swallowing and breathing switching device according to the fifth exemplary embodiment.

As shown in FIG. 7, a swallowing and breathing switching device 10D according to the fifth embodiment is different from the swallowing and breathing switching device 10 according to the first embodiment in that the swallowing and breathing switching device 10D includes a drive signal generating unit 31D. The other configurations of the swallowing and breathing switching device 10D are the same as those of the swallowing and breathing switching device 10, and description of the same parts will be omitted.

The swallowing and breathing switching device 10D includes the drive signal generating unit 31D. The drive signal generating unit 31D is different from the drive signal generating unit 31 in that the switch control circuit 310 is omitted.

In the drive signal generating unit 31D, the electric switch 312 and the electric switch 313 are directly connected to the physical switch 39. The electric switch 312 and the electric switch 313 switch between an open state and a short-circuit state in response to an operation signal from the physical switch 39.

With this configuration, the swallowing and breathing switching device 10D can be configured to switch between a state in which the entrance of the trachea is not blocked and a state in which the entrance of the trachea is blocked by directly reflecting the operation of the wearer.

### Sixth Exemplary Embodiment

An artificial epiglottis and a swallowing and breathing switching device according to a sixth exemplary embodiment will be described with reference to the drawings. FIG. 8 is a view showing a configuration of the swallowing and breathing switching device according to the sixth exemplary embodiment.

As shown in FIG. 8, a swallowing and breathing switching device 10E according to the sixth embodiment is different from the swallowing and breathing switching device 10 according to the first embodiment in that, in the swallowing and breathing switching device 10E, the physical switch 39 is omitted and a myoelectric sensor 51 and a control signal generating unit 52 are provided. The other configurations of the swallowing and breathing switching device 10E are the same as those of the swallowing and breathing switching device 10, and descriptions of the same parts will be omitted.

The swallowing and breathing switching device 10E includes the myoelectric sensor 51 and the control signal generating unit 52. The myoelectric sensor 51 is disposed in the oral cavity of the body on which the artificial epiglottis 20 is worn. The myoelectric sensor 51 detects the movement of the muscles of the oral cavity and generates a myoelectric detection signal. The myoelectric sensor 51 outputs the myoelectric detection signal to the control signal generating unit 52.

The control signal generating unit 52 stores the myoelectric detection signal during swallowing and the myoelectric detection signal during breathing in advance in a memory. The control signal generating unit 52 may alternatively store at least the myoelectric detection signal during swallowing in the memory.

If the myoelectric detection signal from the myoelectric sensor 51 and the myoelectric detection signal during swallowing are the same, the control signal generating unit 52 outputs a control signal indicating a swallowing state to the switch control circuit 310. When the control signal indicating the swallowing state is received, the switch control circuit 310 generates a switch control signal for performing a control to short-circuit the electric switch 312 and the electric switch 313. The switch control circuit 310 outputs the switch control signal to the electric switch 312 and the electric switch 313.

When the electric switch 312 and the electric switch 313 are short-circuited by the switch control signal, a voltage is applied to the artificial epiglottis 20. Therefore, the artificial epiglottis 20 becomes a second shape in which the entrance of the trachea is blocked.

On the other hand, if the myoelectric detection signal from the myoelectric sensor 51 and the myoelectric detection signal during swallowing are not the same, the control signal generating unit 52 does not output the control signal to the switch control circuit 310. When the control signal is not received from the control signal generating unit 52, the switch control circuit 310 does not generate a switch control signal for performing a control to short-circuit the electric switch 312 and the electric switch 313.

When the electric switch 312 and the electric switch 313 are opened, no voltage is applied to the artificial epiglottis 20. Therefore, the artificial epiglottis 20 becomes a first shape in which the entrance of the trachea is not blocked.

With the above-described configuration and control, the swallowing and breathing switching device 10E can be configured to switch between a state in which the entrance of the trachea is not blocked and a state in which the entrance of the trachea is blocked, in the same manner as the swallowing and breathing switching device 10, so that aspiration is suppressed more reliably.

Further, the swallowing and breathing switching device 10E can be configured to switch between a state in which the entrance of the trachea is not blocked and a state in which the entrance of the trachea is blocked in response to the mouth movement of the wearer of the artificial epiglottis 20. Therefore, the swallowing and breathing switching device 10E can suppress aspiration more reliably.

### Seventh Exemplary Embodiment

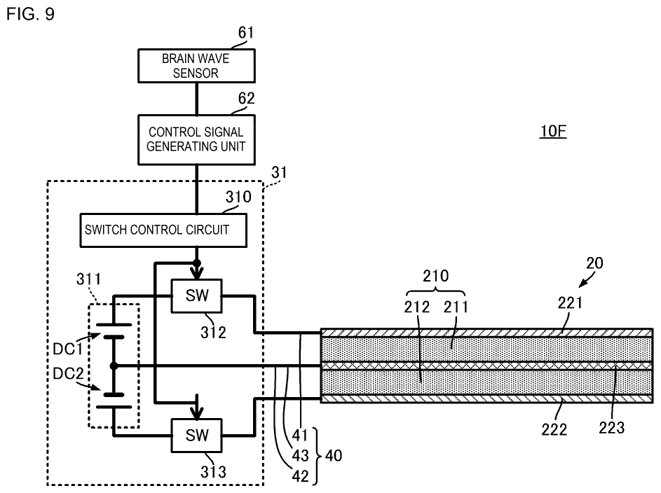

An artificial epiglottis and a swallowing and breathing switching device according to a seventh exemplary embodiment will be described with reference to the drawings. FIG. 9 is a view showing a configuration of the swallowing and breathing switching device according to the seventh exemplary embodiment.

As shown in FIG. 9, a swallowing and breathing switching device 10F according to the seventh embodiment is different from the swallowing and breathing switching device 10 according to the first embodiment in that, in the swallowing and breathing switching device 10F, the physical switch 39 is omitted and a brain wave sensor 61 and a control signal generating unit 62 are provided. The other configurations of the swallowing and breathing switching device 10F are the same as those of the swallowing and breathing switching device 10, and descriptions of the same parts will be omitted.

The swallowing and breathing switching device 10F includes the brain wave sensor 61 and the control signal generating unit 62. The brain wave sensor 61 is disposed on the head on which the artificial epiglottis 20 is worn. The brain wave sensor 61 detects a brain wave and generates a brain wave detection signal. The brain wave sensor 61 outputs the brain wave detection signal to the control signal generating unit 62.

The control signal generating unit 62 stores the brain wave detection signal during swallowing and the brain wave detection signal during breathing in advance in a memory. The control signal generating unit 62 may alternatively store at least the brain wave detection signal during swallowing in the memory.

If the brain wave detection signal from the brain wave sensor 61 and the brain wave detection signal during swallowing are the same, the control signal generating unit 62 is configured to output a control signal indicating the swallowing state to the switch control circuit 310. When the control signal indicating the swallowing state is received, the switch control circuit 310 is configured to generate a switch control signal for performing a control to short-circuit the electric switch 312 and the electric switch 313. The switch control circuit 310 is then configured to output the switch control signal to the electric switch 312 and the electric switch 313.

When the electric switch 312 and the electric switch 313 are short-circuited by the switch control signal, a voltage is applied to the artificial epiglottis 20. Therefore, the artificial epiglottis 20 becomes a second shape in which the entrance of the trachea is blocked.

On the other hand, if the myoelectric detection signal from the brain wave sensor 61 and the myoelectric detection signal during swallowing are not the same, the control signal generating unit 62 does not output the control signal to the switch control circuit 310. When the control signal is not received from the control signal generating unit 62, the switch control circuit 310 does not generate a switch control signal for performing a control to short-circuit the electric switch 312 and the electric switch 313.

When the electric switch 312 and the electric switch 313 are opened, no voltage is applied to the artificial epiglottis 20. Therefore, the artificial epiglottis 20 becomes a first shape in which the entrance of the trachea is not blocked.

With the above-described configuration and control, the swallowing and breathing switching device 10F can be configured to switch between a state in which the entrance of the trachea is not blocked and a state in which the entrance of the trachea is blocked, in the same manner as the swallowing and breathing switching device 10, so that aspiration is suppressed more reliably.

Further, the swallowing and breathing switching device 10F can be configured to switch between a state in which the entrance of the trachea is not blocked and a state in which the entrance of the trachea is blocked in response to the brain wave of the wearer of the artificial epiglottis 20 during swallowing and during breathing. Therefore, the swallowing and breathing switching device 10F suppresses aspiration more reliably.

### Eighth Exemplary Embodiment

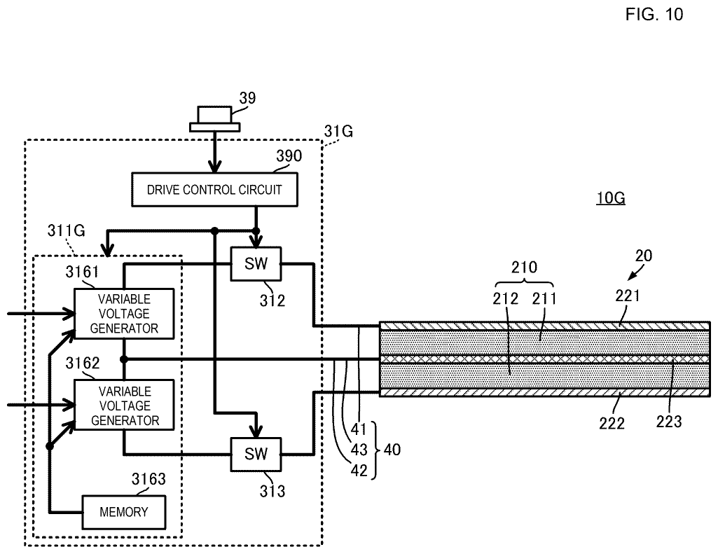

An artificial epiglottis and a swallowing and breathing switching device according to an eighth exemplary embodiment will be described with reference to the drawings. FIG. 10 is a view showing a configuration of the swallowing and breathing switching device according to the eighth exemplary embodiment.

As shown in FIG. 10, a swallowing and breathing switching device 10G according to the eighth embodiment is different from the swallowing and breathing switching device 10 according to the first embodiment in that the swallowing and breathing switching device 10G includes a drive signal generating unit 31G. The other configurations of the swallowing and breathing switching device 10G are the same as those of the swallowing and breathing switching device 10, and descriptions of the same parts will be omitted.

The swallowing and breathing switching device 10G includes the drive signal generating unit 31G. The drive signal generating unit 31G is different from the drive signal generating unit 31 according to the first embodiment in that the drive signal generating unit 31G includes a power supply circuit 311G and a drive control circuit 390. The other configurations of the drive signal generating unit 31G are the same as those of the drive signal generating unit 31, and descriptions of the same parts will be omitted.

The power supply circuit 311G includes a variable voltage generator 3161, a variable voltage generator 3162, and a memory 3163. In an exemplary aspect, the variable voltage generator 3161 and the variable voltage generator 3162 are programmable variable voltage devices, and the voltage value of the direct-current voltage to be outputted can be set.

The memory 3163 is configured to store a voltage value corresponding to the wearer. More specifically, the memory 3163 stores the relationship between the bending angle of the artificial epiglottis 20 and the voltage corresponding to the bending angle for the wearer of the artificial epiglottis 20.

The drive control circuit 390 is configured to control the variable voltage generator 3161 and the variable voltage generator 3162 based on the relationship between the bending angle of the artificial epiglottis 20 and the voltage corresponding to the bending angle. Based on the control from the drive control circuit 390, the variable voltage generator 3161 and the variable voltage generator 3162 are configured to generate a direct-current voltage corresponding to the voltage value stored in the memory 3163.

The drive control circuit 390 is configured to control the drive of the variable voltage generator 3161 and the variable voltage generator 3162, and to control the opening and short-circuiting of the electric switch 312 and the electric switch 313. The control of the electric switch 312 and the electric switch 313 by the drive control circuit 390 is similar to the control of the switch control circuit 310 described above.

With the above-described configuration and control, the swallowing and breathing switching device 10G can be configured to switch between a state in which the entrance of the trachea is not blocked and a state in which the entrance of the trachea is blocked, in the same manner as the swallowing and breathing switching device 10, so that aspiration is suppressed more reliably.

Further, the swallowing and breathing switching device 10G can be configured to control the voltage value of the voltage applied to the artificial epiglottis 20 by the variable voltage generator 3161 and the variable voltage generator 3162. Thus, the bending amount of the artificial epiglottis 20 can be adjusted according to the wearer. Therefore, the swallowing and breathing switching device 10G suppresses aspiration more reliably.

### Ninth Exemplary Embodiment

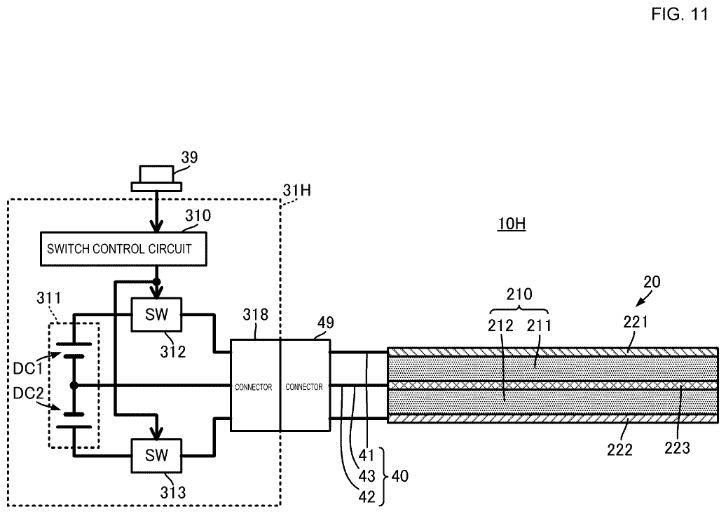

An artificial epiglottis and a swallowing and breathing switching device according to a ninth exemplary embodiment will be described with reference to the drawings. FIG. 11 is a view showing a configuration of the swallowing and breathing switching device according to the ninth exemplary embodiment.

As shown in FIG. 11, a swallowing and breathing switching device 10H according to the ninth embodiment is different from the swallowing and breathing switching device 10 according to the first embodiment in that the swallowing and breathing switching device 10H includes a connector 318 and a connector 49. The other configurations of the swallowing and breathing switching device 10H are the same as those of the swallowing and breathing switching device 10, and descriptions of the same parts will be omitted.

The swallowing and breathing switching device 10H includes a drive signal generating unit 31H. The drive signal generating unit 31H is different from the drive signal generating unit 31 according to the first embodiment in that the drive signal generating unit 31H includes the connector 318. The other configurations of the drive signal generating unit 31H are the same as those of the drive signal generating unit 31, and descriptions of the same parts will be omitted.

The connector 318 is connected to the electric switch 312, the electric switch 313, and the node of the direct-current power supply DC1 and direct-current power supply DC2 in the power supply circuit 311.

The connector 49 is connected to a cable 40 (a first cable 41, a second cable 42, and a third cable 43).

The connector 318 and the connector 49 are detachably connected to each other. When the connector 318 and the connector 49 are connected, the electric switch 312 is connected to the first cable 41, and the electric switch 313 is connected to the second cable 42. The node of the direct-current power supply DC1 and direct-current power supply DC2 in the power supply circuit 311 are connected to the third cable 43.

With such a configuration, the swallowing and breathing switching device 10H can be configured to switch between a state in which the entrance of the trachea is not blocked and a state in which the entrance of the trachea is blocked, in the same manner as the swallowing and breathing switching device 10, so that aspiration is suppressed more reliably.

Further, in the swallowing and breathing switching device 10H, the drive signal generating unit 31H can be replaced. Therefore, the swallowing and breathing switching device 10H is more user-friendly for the wearer.

### Tenth Exemplary Embodiment

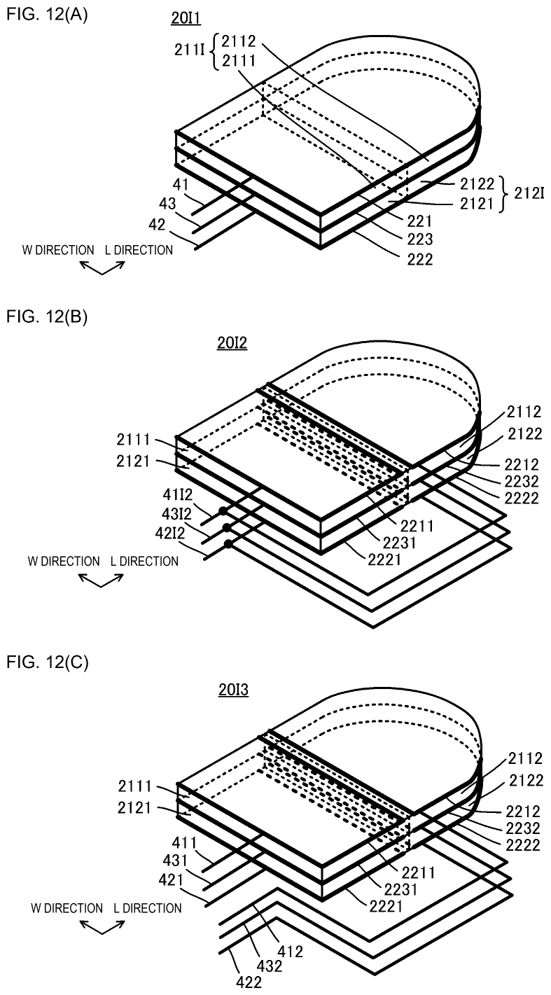

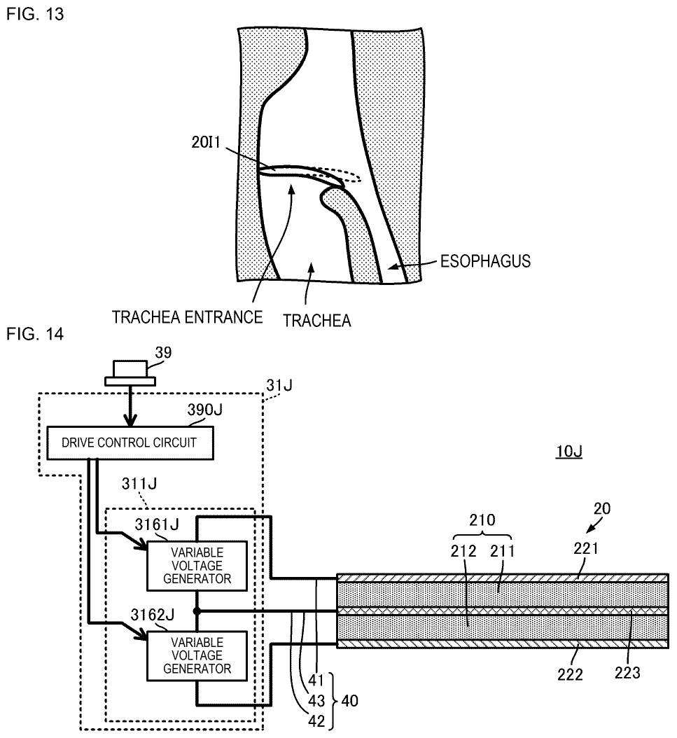

An artificial epiglottis according to a tenth exemplary embodiment will be described with reference to the drawings. FIG. 12(A), FIG. 12(B), and FIG. 12(C) are perspective views each showing a configuration example of the artificial epiglottis according to the tenth exemplary embodiment. FIG. 13 is a schematic view showing a curved state of the artificial epiglottis shown in FIG. 12(A). The dotted line shown in FIG. 13 is a schematic view showing an example of a case in which there are no plurality of portions with different piezoelectric constants as shown in FIG. 12(A) (i.e., there is one portion with one piezoelectric constant).

As shown in FIG. 12(A), an artificial epiglottis 20I1 according to the tenth embodiment is different from the artificial epiglottis 20 according to the first embodiment in that, in the artificial epiglottis 20I1, the piezoelectric element is composed of a plurality of portions with different piezoelectric constants. The basic configuration of the artificial epiglottis 20I1 is the same as that of the artificial epiglottis 20, and descriptions of the same parts will be omitted.

The artificial epiglottis 20I1 includes a first piezoelectric element 211I and a second piezoelectric element 212I.

In this exemplary aspect, the first piezoelectric element 211I includes a first portion 2111 and a second portion 2112. The first portion 2111 and the second portion 2112 are arranged in the order of the first portion 2111 and the second portion 2112 from one end (e.g., a fixed end to be fixed to the pharynx) to the other end (e.g., a movable end) of the first piezoelectric element 211I (i.e., in an L direction in the drawing).

The piezoelectric constant of the second portion 2112 is larger than that of the first portion 2111.

The second piezoelectric element 212I includes a third portion 2121 and a fourth portion 2122. The third portion 2121 and the fourth portion 2122 are arranged in the order of the third portion 2121 and the fourth portion 2122 from one end (a fixed end to be fixed to the pharynx) to the other end (a movable end) of the second piezoelectric element 212I (i.e., in the L direction in the drawing).

The piezoelectric constant of the fourth portion 2122 is larger than that of the third portion 2121. The piezoelectric constant of the fourth portion 2122 is the same as that of the second portion 2112, and the piezoelectric constant of the third portion 2121 is the same as that of the first portion 2111.

The first driving electrode 221 overlaps the first portion 2111 and the second portion 2112. The second driving electrode 222 overlaps the third portion 2121 and the fourth portion 2122. The third driving electrode 223 is disposed between the first portion 2111 and the third portion 2121 and is also disposed between the second portion 2112 and the fourth portion 2122.

Thus, the same voltage is applied to the first laminated portion where the first portion 2111 and the third portion 2121 are laminated, and the second laminated portion where the second portion 2112 and the fourth portion 2122 are laminated.

The piezoelectric constant of the second laminated portion where the second portion 2112 and the fourth portion 2122 are laminated is larger than the piezoelectric constant of the first laminated portion where the first portion 2111 and the third portion 2121 are laminated.

Therefore, as shown in FIG. 13, the second laminated portion is curved more than the first laminated portion.

Here, when the tracheal entrance and the esophagus are small, as shown by the dotted line in FIG. 13, the artificial epiglottis that does not have a plurality of piezoelectric constants may block a part of the esophagus or make it difficult to bring the artificial epiglottis into close contact with the tracheal entrance.

However, the artificial epiglottis 20I1 has its portion on the movable end side curved more than its portion on the fixed end side. This configuration allows the opening area of the esophagus to be secured more reliably and allows the artificial epiglottis 20I1 to be brought into close contact with the tracheal entrance more reliably.

At this time, the curvature of the artificial epiglottis 20I1 is configured to be adjusted by adjusting the voltage. Therefore, the artificial epiglottis 20I1 secures the opening area of the esophagus more reliably according to the wearer, and the artificial epiglottis 20I1 is brought into close contact with the tracheal entrance more reliably.

As shown in FIG. 12(B), an artificial epiglottis 20I2 is different from the artificial epiglottis 20I1 in the configuration of the driving electrodes. The other configurations of the artificial epiglottis 20I2 are the same as those of the artificial epiglottis 20I1, and description of the similar parts will be omitted.

In this exemplary aspect, the artificial epiglottis 20I2 includes a first driving electrode 2211, a second driving electrode 2221, a third driving electrode 2231, a fourth driving electrode 2212, a fifth driving electrode 2222, and a sixth driving electrode 2232.

The first driving electrode 2211 and the fourth driving electrode 2212 are arranged side by side separated from each other in an L direction of the artificial epiglottis 20I2. The second driving electrode 2222 and the fifth driving electrode 2212 are arranged side by side separated from each other in the L direction of the artificial epiglottis 20I2. The third driving electrode 2231 and the sixth driving electrode 2232 are arranged side by side separated from each other in the L direction of the artificial epiglottis 20I2.

The first driving electrode 2211 and the third driving electrode 2231 are disposed with the first portion 2111 of the first piezoelectric element 211I sandwiched therebetween. The fourth driving electrode 2212 and the sixth driving electrode 2232 are disposed with the second portion 2112 of the first piezoelectric element 211I sandwiched therebetween.

The second driving electrode 2221 and the third driving electrode 2231 are disposed with the third portion 2121 of the second piezoelectric element 212I sandwiched therebetween. The fifth driving electrode 2222 and the sixth driving electrode 2232 are disposed with the fourth portion 2122 of the second piezoelectric element 212I sandwiched therebetween.

The first driving electrode 2211 and the fourth driving electrode 2212 are connected to a first cable 41I2. The second driving electrode 2221 and the fifth driving electrode 2222 are connected to a second cable 42I2. The third driving electrode 2231 and the sixth driving electrode 2232 are connected to a third cable 43I2.

With such a configuration, the artificial epiglottis 20I2 exhibits the same effects as the artificial epiglottis 20I1.

As shown in FIG. 12(C), an artificial epiglottis 20I3 is different from the artificial epiglottis 20I2 in the configuration of the cables connected to the driving electrodes. The other configurations of the artificial epiglottis 20I3 are the same as those of the artificial epiglottis 20I2, and descriptions of the same parts will be omitted.

The artificial epiglottis 20I3 includes a first cable 411, a second cable 421, a third cable 431, a fourth cable 412, a fifth cable 422, and a sixth cable 432.

The first cable 411 is connected to the first driving electrode 2211, and the fourth cable 412 is connected to the fourth driving electrode 2212. The second cable 421 is connected to the second driving electrode 2221, and the fifth cable 422 is connected to the fifth driving electrode 2222. The third cable 431 is connected to the third driving electrode 2231, and the sixth cable 432 is connected to the sixth driving electrode 2232.

With such a configuration, the artificial epiglottis 20I3 exhibits the same effects as the artificial epiglottises 20I1 and 20I2.

Further, in the artificial epiglottis 20I3, it is possible to apply different voltages to the portion on the fixed end side and to the portion on the movable end side. Therefore, in the artificial epiglottis 20I3, the amount of curvature can be further adjusted. Thus, with the artificial epiglottis 20I3, the shape adjustment according to the wearer can be performed more accurately.

In the present embodiment, a configuration is described in which a plurality of portions having different piezoelectric constants are disposed along the L direction of the artificial epiglottis. However, it is also possible to provide a configuration in which a plurality of portions having different piezoelectric constants are disposed along a W direction (a direction orthogonal to the L direction) of the artificial epiglottis. Further, the number of the portions having different piezoelectric constants is not limited to 2 but may be 3 or more.

### Eleventh Exemplary Embodiment

An artificial epiglottis and a swallowing and breathing switching device according to an eleventh exemplary embodiment will be described with reference to the drawings. FIG. 14 is a view showing a configuration of the swallowing and breathing switching device according to the eleventh exemplary embodiment.

As shown in FIG. 14, a swallowing and breathing switching device 10J according to the eleventh embodiment is different from the swallowing and breathing switching device 10 according to the first embodiment in that the swallowing and breathing switching device 10J includes a drive signal generating unit 31J. The other configurations of the swallowing and breathing switching device 10J are the same as those of the swallowing and breathing switching device 10, and descriptions of the same parts will be omitted.

The drive signal generating unit 31J includes a drive control circuit 390J and a power supply circuit 311J. The power supply circuit 311J includes a variable voltage generator 3161J and a variable voltage generator 3162J.

The drive control circuit 390J is configured to control, based on the depressed state and the depressed history of the physical switch 39, the output voltages of the variable voltage generator 3161J and the variable voltage generator 3162J, that is, the voltages supplied to the artificial epiglottis 20.

For example, when detecting that the physical switch 39 is pressed, the drive control circuit 390J is configured to perform an output control of a first state. In the output control of the first state, the drive control circuit 390J performs a control to output +3V to the variable voltage generator 3161J and output −3V to the variable voltage generator 3162J.

Next, when detecting that the physical switch 39 is pressed again, the drive control circuit 390J is configured to perform an output control of a second state. In the output control of the second state, the drive control circuit 390J is configured to perform a control to output −3V to the variable voltage generator 3161J and output +3V to the variable voltage generator 3162J.

Next, when detecting that the physical switch 39 is pressed again, the drive control circuit 390J is configured to perform the output control of the first state. In the output control of the first state, the drive control circuit 390J is configured to perform a control to output +3V to the variable voltage generator 3161J and output −3V to the variable voltage generator 3162J.

Hereinafter, the drive control circuit 390J repeats output control of the first state and output control of the second state as described above.

With such a configuration, the swallowing and breathing switching device 10J can easily control the voltages supplied to the artificial epiglottis 20. Further, the swallowing and breathing switching device 10J can shape the artificial epiglottis 20 as desired by the wearer at a time desired by the wearer.

In general, it is noted that the configurations of the embodiments described above can be suitably combined, and effects corresponding to the respective combinations can be achieved. For example, the configuration of the swallowing and breathing switching device 10E according to the sixth embodiment and the configuration of the swallowing and breathing switching device 10F according to the seventh embodiment can be combined. The swallowing and breathing switching device obtained by such a combination can switch between a state in which the entrance of the trachea is not blocked and a state in which the entrance of the trachea is blocked by using the brain wave and the myoelectricity. Therefore, aspiration is suppressed more reliably.

Next, the features of the exemplary embodiments according to the present disclosure will be collectively described.

In an exemplary aspect <1>, an artificial epiglottis is provided comprising: a membrane-like piezoelectric element configured to bend depending on a voltage; and a driving electrode that applies the voltage to the piezoelectric element. The piezoelectric element is structurally configured to be disposed on a larynx, and the piezoelectric element is configured to becomes a first shape in which an entrance of a trachea is blocked or a second shape in which the entrance of the trachea is opened, depending on the voltage.

In an exemplary aspect <2>, for the artificial epiglottis according to exemplary aspect <1>, the piezoelectric element includes a plurality of piezoelectric elements, the plurality of piezoelectric elements are laminated in a state in which membrane surfaces thereof are parallel to each other, and the plurality of piezoelectric elements generate synergized bending depending on the voltage.

In an exemplary aspect <3>, for the artificial epiglottis according to exemplary aspect <2>, the plurality of piezoelectric elements are composed of a first piezoelectric element and a second piezoelectric element, and the first piezoelectric element and the second piezoelectric element have opposite displacement directions when the voltage is applied.

In an exemplary aspect <4>, a swallowing and breathing switching device is provide that includes the artificial epiglottis according to any one of exemplary aspects <1> to <3>; and a drive signal generating unit configured to generate a drive signal generating the voltage.

In an exemplary aspect <5>, for the swallowing and breathing switching device according to exemplary aspect <4>, the drive signal generating unit switches between a first state of the drive signal and a second state of the drive signal, and the artificial epiglottis switches between the first shape or the second shape depending on the first state and the second state.

In an exemplary aspect <6>, for the swallowing and breathing switching device according to exemplary aspect <5>, the drive signal generating unit includes an electric switch, and switches between the first state and the second state depending on opening and short-circuiting of the electric switch.

In an exemplary aspect <7>, the swallowing and breathing switching device according to exemplary aspect <6>, further includes a physical switch that controls the electric switch.

In an exemplary aspect <8>, for the swallowing and breathing switching device according to exemplary aspect <5>, the drive signal generating unit includes an alternating-current power supply, and switches between the first state and the second state depending on whether the voltage from the alternating-current power supply is positive or negative.

In an exemplary aspect <9>, for the swallowing and

breathing switching device according to any one of exemplary aspects <4> to <8>, the drive signal generating unit is disposed outside a body on which the artificial epiglottis is worn, and the swallowing and breathing switching device further comprises a cable that connects the drive signal generating unit and the artificial epiglottis.

In an exemplary aspect <10>, for the swallowing and breathing switching device according to any one of exemplary aspects <4> to <8>, the drive signal generating unit is disposed outside a body on which the artificial epiglottis is worn, and the drive signal generating unit comprises: a power transmission coil disposed outside the body; and a power reception coil disposed inside the body, and connected to the artificial epiglottis.

In an exemplary aspect <11>, the swallowing and breathing switching device according to exemplary aspect <6>, further comprises: a myoelectric sensor that detects movement of muscles of an oral cavity of a body on which the artificial epiglottis is worn, and generates a myoelectric detection signal; and a switch control circuit that controls the opening and short-circuiting of the electric switch based on the myoelectric detection signal.

In an exemplary aspect <12>, the swallowing and breathing switching device according to exemplary aspect <11>, further comprises: a memory that stores a relationship between the myoelectric detection signal and swallowing and breathing; and a switch control circuit that controls the opening and short-circuiting of the electric switch based on the relationship between the myoelectric detection signal and swallowing and breathing stored in the memory.

In an exemplary aspect <13>, the swallowing and breathing switching device according to exemplary aspect <6>, further comprises: a brain wave sensor that detects a brain wave of a body on which the artificial epiglottis is worn, and generates a brain wave detection signal; and a switch control circuit that controls the opening and short-circuiting of the electric switch based on the brain wave detection signal.

In an exemplary aspect <14>, the swallowing and breathing switching device according to exemplary aspect <13>, further comprises: a memory that stores a relationship between the brain wave detection signal and swallowing and breathing; and a switch control circuit that controls the opening and short-circuiting of the electric switch based on the relationship between the brain wave detection signal and swallowing and breathing stored in the memory.

In an exemplary aspect <15>, for the swallowing and breathing switching device according to any one of exemplary aspects <4> to <14>, the drive signal generating unit includes a programmable variable voltage device.

In an exemplary aspect <16>, the swallowing and breathing switching device according to exemplary aspect <15>, further includes: a memory that stores a relationship between a bending angle of the artificial epiglottis for a wearer of the artificial epiglottis and a voltage corresponding to the bending angle; and a drive control circuit that controls an output voltage of the programmable variable voltage device. In this aspect, the drive control circuit controls the output voltage of the programmable variable voltage device based on the relationship between the bending angle of the artificial epiglottis for the wearer of the artificial epiglottis and the voltage corresponding to the bending angle stored in the memory.

### REFERENCE SIGNS LIST

10, 10A, 10B, 10C, 10D, 10E, 10F, 10G, 10H, 10J swallowing and breathing switching device
20, 20A, 20B, 20I1, 20I2, 20I3 artificial epiglottis
31, 31A, 31B, 31C, 31D, 31G, 31H drive signal generating unit
32 orthosis
39 physical switch
40, 40A, 40B cable
41 first cable
42 second cable
43 third cable
49 connector
51 myoelectric sensor
52 control signal generating unit
61 brain wave sensor
62 control signal generating unit
211, 211I first piezoelectric element
2111 first portion
2122 second portion
212, 212I second piezoelectric element
2121 third portion
2122 fourth portion
221, 2211 first driving electrode
222, 2221 second driving electrode
223, 2231 third driving electrode
2212 fourth driving electrode
2222 fifth driving electrode
2232 sixth driving electrode
310 switch control circuit
311, 311A, 311B, 311C, 311G, 311J power supply circuit
312, 313 electric switch
314 power transmission control unit
315 power transmission coil
316 power reception coil
317 power reception control unit
318 connector
390, 390J drive control circuit
41, 41I2, 411 first cable
42, 42I2, 421 second cable
43, 43I2, 431 third cable
412 fourth cable
422 fifth cable
432 sixth cable
3161, 3162, 3161J, 3162J variable voltage generator
3163 memory

AC alternating-current power supply
C20 cover
DC1, DC10, DC2 direct-current power supply
H31 housing

What is claimed:

1. An artificial epiglottis comprising:
a membrane-like piezoelectric element configured to bend in response to a voltage applied thereto; and
a driving electrode configured to apply the voltage to the piezoelectric element,
wherein the piezoelectric element is structurally configured to be disposed on a larynx, and
wherein the piezoelectric element is configured to bend, in response to the voltage, to become a first shape to block an entrance of a trachea and to become a second shape to open the entrance of the trachea.

2. The artificial epiglottis according to claim 1, wherein:
the piezoelectric element includes a plurality of piezoelectric elements that are laminated in a state in which membrane surfaces thereof are parallel to each other, and
the plurality of piezoelectric elements are configured to generate synergized bending depending on the voltage applied by the driving electrode.

3. The artificial epiglottis according to claim 2, wherein the plurality of piezoelectric elements include a first piezoelectric element and a second piezoelectric element that have opposite displacement directions from each other when the voltage is applied thereto.

4. A swallowing and breathing switching device comprising:
the artificial epiglottis according to claim 1; and
a drive signal generating unit configured to generate a drive signal generating the voltage.

5. The swallowing and breathing switching device according to claim 4, wherein:
the drive signal generating unit is configured to switch between a first state of the drive signal and a second state of the drive signal, and
the artificial epiglottis is configured to switch between the first shape or the second shape depending on the first state and the second state, respectively.

6. The swallowing and breathing switching device according to claim 5, wherein the drive signal generating unit includes an electric switch that is configured to switch between the first state and the second state depending on opening and short-circuiting of the electric switch.

7. The swallowing and breathing switching device according to claim 6, further comprising a physical switch configured to control the electric switch.

8. The swallowing and breathing switching device according to claim 5, wherein the drive signal generating unit includes an alternating-current power supply, and is configured to switch between the first state and the second state depending on whether the voltage from the alternating-current power supply is positive or negative.

9. The swallowing and breathing switching device according to claim 4, wherein:
the drive signal generating unit is disposed outside a body on which the artificial epiglottis is worn, and
the swallowing and breathing switching device further comprises a cable that connects the drive signal generating unit to the artificial epiglottis.

10. The swallowing and breathing switching device according to claim 4, wherein:
the drive signal generating unit is disposed outside a body on which the artificial epiglottis is worn, and
the drive signal generating unit comprises:
a power transmission coil disposed outside the body; and
a power reception coil disposed inside the body, and connected to the artificial epiglottis.

11. The swallowing and breathing switching device according to claim 6, further comprising:
a myoelectric sensor configured to detect a movement of muscles of an oral cavity of a body on which the artificial epiglottis is worn, and to generate a myoelectric detection signal based on the detected movement of muscles; and
a switch control circuit configured to control the opening and short-circuiting of the electric switch based on the myoelectric detection signal.

12. The swallowing and breathing switching device according to claim 11, further comprising:
a memory configured to store a relationship between the myoelectric detection signal and swallowing and breathing; and
a switch control circuit configured to control the opening and short-circuiting of the electric switch based on the relationship between the myoelectric detection signal and swallowing and breathing stored in the memory.

13. The swallowing and breathing switching device according to claim 6, further comprising:
a brain wave sensor configured to detect a brain wave of a body on which the artificial epiglottis is worn, and to generate a brain wave detection signal based on the detected brain wave of the body; and
a switch control circuit configured to control the opening and short-circuiting of the electric switch based on the brain wave detection signal.

14. The swallowing and breathing switching device according to claim 13, further comprising:
a memory configured to store a relationship between the brain wave detection signal and swallowing and breathing; and
a switch control circuit configured to control the opening and short-circuiting of the electric switch based on the relationship between the brain wave detection signal and swallowing and breathing stored in the memory.

15. The swallowing and breathing switching device according to claim 4, wherein the drive signal generating unit includes a programmable variable voltage device.

16. The swallowing and breathing switching device according to claim 15, further comprising:
a memory configured to store a relationship between a bending angle of the artificial epiglottis for a wearer of the artificial epiglottis and a voltage corresponding to the bending angle; and
a drive control circuit configured to control an output voltage of the programmable variable voltage device based on the relationship between the bending angle of the artificial epiglottis for the wearer of the artificial epiglottis and the voltage corresponding to the bending angle stored in the memory.

17. An artificial epiglottis comprising:
a plurality of piezoelectric elements that include a first piezoelectric element and a second piezoelectric element disposed in parallel to each other; and
a plurality of driving electrodes that include a first driving electrode on a surface of the first piezoelectric element, a second driving electrode on a surface of the second piezoelectric element, and a third driving electrode disposed between the first piezoelectric element and the second piezoelectric element,
wherein the plurality of piezoelectric elements are structurally configured to be disposed on a larynx,
wherein the plurality of driving electrodes are configured to apply at least one voltage to the plurality of piezoelectric elements to configure the plurality of piezoelectric elements between a first shape to block an entrance of a trachea and a second shape to open the entrance of the trachea, and
wherein the plurality of piezoelectric elements are configured to generate synergized bending depending on the at least one voltage applied by the plurality of driving electrodes.

18. The artificial epiglottis according to claim 17, wherein:
the first piezoelectric element and the second piezoelectric element comprise a polylactic acid, and
the first piezoelectric element and the second piezoelectric element are configured to extend and contract in a direction parallel to the respective surfaces thereof depending on the at least one voltage applied thereto.

19. A swallowing and breathing switching device comprising:
the artificial epiglottis according to claim 17; and
a drive signal generating unit configured to generate a drive signal generating the voltage.

20. The swallowing and breathing switching device according to claim 19, wherein:
the drive signal generating unit is configured to switch between a first state of the drive signal and a second state of the drive signal, and
the artificial epiglottis is configured to switch between the first shape or the second shape depending on the first state and the second state, respectively.
